(19) United States
(12) Patent Application Publication
(10) Pub. No.: US 2020/0022935 A1
(43) Pub. Date: Jan. 23, 2020

(54) TOPICAL FORMULATION FOR TREATING BRUISING

(71) Applicant: JERRY MCSHANE, DEER PARK, TX (US)

(72) Inventor: JERRY MCSHANE, DEER PARK, TX (US)

(21) Appl. No.: 16/042,966

(22) Filed: Jul. 23, 2018

(51) Int. Cl. A61K 31/194; A61K 9/00; A61K 31/045; A61K 31/34; A61K 31/133; A61P 17/00

(52) U.S. Cl. CPC A61K 31/194; A61K 9/0014; A61P 17/00; A61K 31/34; A61K 31/133; A61K 31/045

(57) ABSTRACT

A topical formulation for treating bruising can include an exfoliant, a protein denaturant, a penetration enhancer, triethanolamine, and water. In an embodiment the topical formulation may include other components selected from a preservative, a detergent, a skin denaturant, and a base. The topical formulation can be particularly effective for reversing bruising caused by Senile Purpura and/or preventing hemosiderin staining.

### BACKGROUND

### 1. Field

The disclosure of the present patent application relates to topical compositions, and particularly to a topical formulation for treating bruising.

### 2. Description of the Related Art

Senile Purpura, sometimes referred to as Batman's Purpura, is a dark blue-black discoloration of the skin caused by either spontaneous or post-traumatic bleeding of, most commonly, the upper extremities and shoulders. Although the size can vary, these discolorations are usually less than five centimeters in diameter and have well demarcated smooth edges. The underlying cause can be traced to the increased friability of the small blood vessels of the arms that results from both photodamage as well as advancing age. This, coupled with the loss of subcutaneous fat and connective tissue, leaves the upper extremities more susceptible to the ill effects of even minor trauma. Post traumatic blood is extravasated into the surrounding dermis producing a dark blue-black discoloration of the skin, which is thought to be due to the iron laden protein hemosiderin.

This natural, age-related, bruising is exacerbated by the increased use of blood thinners, such as Warfin (Coumadin), Aspirin, Xerelto, Pradaxa, Eliquis, Savaysa, Plavix and Cortisteroids. With increased age, this bruising does not always resolve naturally and may progress to the formation of permanent hemosiderin staining. Following a bleed, extravasated red blood cells are deposited into the extravascular space and subsequently die, releasing hemoglobin, the oxygen carrying component of the blood.

Current treatments for Senile Purpura and prevention of hemosiderin staining may include over the counter creams and ointments or natural remedies. Natural remedies are unreliable at best. Over the counter creams and ointments tend to rely on anti-inflammatories, which may not reach the deep tissues involved in hemosiderin staining and may prove ineffective in treating Senile Purpura.

Thus, a topical formulation for treating bruising solving the aforementioned problems is desired.

### SUMMARY

A topical formulation for treating bruising can include an exfoliant, a protein denaturant, a penetration enhancer, triethanolamine, and water. In an embodiment the topical formulation may include one or more other components selected from a preservative, a detergent, a skin denaturant, and a base. The topical formulation can be particularly effective for reversing bruising caused by Senile Purpura and/or preventing hemosiderin staining.

The topical formulation may be combined with any delivery system known in the art, such as an ointment, cream, lotion, liquid, roll-on, patch, gel, paste, micelle, dermal patch, or an occlusion.

An embodiment of the present subject matter is directed to a method of treating bruising, including administering to a patient in need thereof at least one topical formulation according to the present subject matter. The method of treating bruising may include iontophoresis.

These and other features of the present disclosure will become readily apparent upon further review of the following specification and drawings.

### BRIEF DESCRIPTION OF THE DRAWINGS

FIG. 1A shows an arm bruise on day 1 before treatment with the topical formulation for treating bruising according to the present subject matter.

FIG. 1B shows the FIG. 1A bruise after three days of treatment with the topical formulation for treating bruising.

FIG. 1C shows the FIGS. 1A and 1B bruise after five days of treatment with the topical formulation for treating bruising.

FIG. 2A shows an arm bruise on day 1 before treatment with the topical formulation for treating bruising.

FIG. 2B shows the FIG. 2A bruise after three days of treatment with the topical formulation for treating bruising.

FIG. 2C shows the FIGS. 2A and 2B bruise after six days of treatment with the topical formulation for treating bruising.

### DETAILED DESCRIPTION OF THE PREFERRED EMBODIMENTS

A topical formulation for treating bruising can be topically administered to a patient for treating bruising, and particularly, for treating bruising associated with purpura or for preventing hemosiderin staining. The composition can be directly applied to a bruise on the skin. The topical formulation for treating bruising can include an exfoliant, a protein denaturant, a penetration enhancer, triethanolamine, and water. Optionally, the topical formulation can include a humectant, e.g., glycerin, and/or a scented natural oil, e.g., lavender or rosemary. The humectant can mitigate the drying effects of the alcohols in the formulation. The scented oil can provide fragrance as well as reduce the evaporation rate of volatiles.

As used herein to define a weight by volume percentage, the term “about” shall mean within 1.0% of the specified weight by volume percentage.

The exfoliant may be any exfoliant known in the art, including but not limited to citric acid, salicylic acid, glycolic acid, malic acid, tartaric acid, or mechanical exfoliants.

The protein denaturant may be any protein denaturant known in the art, including but not limited to Butanol, pentanol, octanol, other alcohols, or urea.

The penetration enhancer may be any penetration enhancer known in the art, including but not limited to dimethyl isosorbide (DMI), dimethyl sulfoxide, an azone such as laurocapram, a pyrrolidone, an alcohol, an alkanoid such as ethanol or decanol, glycols, an essential oil, a mineral oil, a vegetable oil, a terpene, a terpenoid, an oxazolidinone, or urea.

In an embodiment the exfoliant may be citric acid, the protein denaturant may be butanol (2-butanol), and the penetration enhancer may be DMI. The citric acid may range from about 1.0% to about 10.0% by weight of the topical formulation (e.g., 0.3% to 8% by weight or 1% by weight). The butanol may range from about 0.1% to about 10.0% by weight of the topical formulation. The DMI may range from about 0.5% to about 15.0% by weight of the topical formulation. The water may range from about 5.0% to about 68.0% by weight of the topical formulation. TEA may be added to the formulation in an amount sufficient to adjust the final solution to a pH ranging from about pH 3.8 to about pH 4.2, e.g., pH 4.0.

In an embodiment the topical formulation may include one or more other components selected from a preservative, a detergent, a skin denaturant, and a base.

The preservative may be any preservative known in the art, including but not limited to benzalkonium chloride (BKC) or Germall® Plus, a preservative including 60% by weight propylene glycol, 39.6% by weight diazolidinyl urea (DU) and 0.4% iodopropynyl butylcarbamate (IPBC).

The detergent may be any detergent known in the art, including but not limited to sodium laureth sulfate (SLES), sodium laurel sulfate, ammonium lauryl sulfate, sodium pareth sulfate, magnesiumlaureth sulfate, or an alkylbenzene sulfonate.

The skin denaturant may be any skin denaturant known in the art, including but not limited to isopropyl alcohol (IPA), a fatty ester, a fatty alcohol, isopropyl mystirate, methanol, or ethanol.

In an embodiment, the detergent may be SLES ranging from about 0.01% to about 10.0% by weight of the topical formulation.

In an embodiment, the skin denaturant may be isopropyl alcohol (IPA). The IPA may range from about 1.0% by weight to about 70.0% by weight of the topical formulation.

In an embodiment, the topical formulation may include at least one preservative. For example, the topical formulation may include about 0.5% by weight Germall® Plus, or about 0.5% by weight BKC.

The composition can be directly administered to a bruised site on the skin of the patient. Once applied on the skin, it is preferable to apply a bandage or other suitable dressing over the composition for occlusion. The dressing can be sterile or non-sterile. A particular formulation of the present composition selected for treatment may be based upon a patient's skin sensitivity classification. The administration may include iontophoresis. It should be understood that gamma radiation of a dressing including the composition may be employed instead of or in addition to including a preservative in the composition.

In an embodiment, the composition can include a formulation suitable for most patients, regardless of their skin sensitivity classification, herein “general skin formulation.”

A first embodiment of the general skin formulation may include about 1.0% by weight citric acid, about 0.3% by weight butanol, about 30.0% by weight IPA, about 10.0% by weight DMI, and about 0.5% by weight of a preservative, preferably Germall® Plus. The composition may be adjusted to a pH of about 4.0 by adding TEA, e.g., about 0.40% by weight TEA. A remainder of the topical composition may be water. The general formulation can be applied to the skin and occluded, as described previously.

A second embodiment of the general skin formulation may include about 1.0% by weight citric acid, about 0.2% by weight butanol, about 5.0% by weight IPA, about 10.0% by weight DMI, and about 0.5% by weight of a preservative, preferably Germall® Plus. The composition may be adjusted to a pH of about 4.0 by adding TEA, e.g., about 0.40% by weight TEA. A remainder of the topical composition may be water. The general formulation can be applied to the skin and occluded, as described previously.

In an embodiment, the composition can include a formulation particularly suitable for patients suffering from sensitive skin, herein “sensitive skin formulation.” Patients identified as having “sensitive skin,” according to the present teachings, typically have skin that is not very dry and generally smooth to the touch. These patients generally tolerate most topical compositions, but may have a history of occasional negative reactions to topical products containing harsh sensitizing chemicals. These patients typically do not suffer from itchy skin.

A first embodiment of the sensitive skin formulation may include about 1.0% by weight citric acid, about 0.5% by weight 2-butanol, about 0.5% by weight SLES, about 20.0% by weight IPA, about 10.0% by weight DMI, and about 0.5% by weight of a preservative, preferably German® Plus. The composition may be adjusted to a pH of about 4.0 using TEA. A remainder of the topical composition may be water. Preferably, this embodiment of the sensitive skin formulation is used with a non-sterile dressing.

A second embodiment of the sensitive skin formulation may include about 1.0% by weight citric acid, about 0.5% by weight 2-butanol, about 0.5% by weight SLES, about 20.0% by weight IPA, and about 10.0% by weight DMI. This composition may be adjusted to a pH of about 4.0 using TEA. A remainder of the topical composition may be water. Preferably, this embodiment of the sensitive skin formulation is used with a sterile dressing.

In an embodiment, the composition can include a formulation particularly suitable for patients suffering from non-sensitive skin, herein “non-sensitive skin formulation.” Patients identified as having “non-sensitive skin,” according to the present teachings, typically have normal, well-hydrated, smooth skin that is soft and smooth to touch. These patients typically do not suffer from itchy or flaky skin.

A first embodiment of the non-sensitive skin formulation may include about 10.0% by weight citric acid, about 10.0% by weight 2-butanol, about 1.0% by weight SLES, and about 15.0% by weight DMI. This composition may be adjusted to a pH of about 4.0 using TEA. A remainder of the topical composition may be water. Preferably, this embodiment of the non-sensitive skin formulation is used with a sterile dressing.

A second embodiment of the non-sensitive skin formulation may include about 10.0% by weight citric acid, about 10.0% by weight 2-butanol, about 1.0% by weight SLES, about 15.0% by weight DMI, and about 0.5% by weight of a preservative, preferably Germall® Plus. This composition may be adjusted to a pH of about 4.0 using TEA. A remainder of the topical composition may be water. Preferably, this embodiment of the non-sensitive skin formulation is used with a non-sterile dressing.

In an embodiment, the composition can include a formulation particularly suitable for patients suffering from very sensitive skin, herein “very sensitive skin formulation.” Patients identified as having “very sensitive skin,” according to the present teachings, typically have dry, flaky, itchy skin, with patches of redness. These patients generally have a history of sensitivity to topical products, such as soaps, make-up removers, and other topical skin care products.

A first embodiment of the very sensitive skin formulation may include about 1.0% by weight citric acid, about 0.2% by weight 2-butanol, about 20.0% by weight IPA, about 10.0% by weight DMI, and about 0.5% by weight of a preservative, preferably Germall® Plus. This composition may be adjusted to a pH of about 4.0 using TEA. A remainder of the topical composition may be water. Preferably, this embodiment of the very sensitive skin formulation is used with a non-sterile dressing.

A second embodiment of the very sensitive skin formulation may include about 1.0% by weight citric acid, about 0.2% by weight 2-butanol, about 20.0% by weight IPA, and about 10.0% by weight DMI. This composition may be adjusted to a pH of about 4.0 using TEA. A remainder of the topical composition may be water. Preferably, this embodiment of the very sensitive skin formulation is used with a sterile dressing.

A third embodiment of the very sensitive skin formulation may be in ointment forms and may include about 1.0% by weight citric acid, about 0.2% by weight 2-butanol, about 20.0% by weight IPA, about 10.0% by weight DMI, about 0.5% by weight of a preservative, preferably Germall® Plus, and about 5.0% by weight water. This composition may be adjusted to a pH of about 4.0 using TEA. A remainder of the topical composition may be an appropriate base composition. The appropriate base composition may be any base ointment suitable for delivering an ointment.

In an embodiment, the base ointment may be AquaBASE Ointemént. AquaBASE Ointment may include petrolatum, mineral oil, mineral wax, wool wax alcohol, and cholesterol. Preferably, this embodiment of the very sensitive skin formulation is used with a non-sterile dressing.

According to an embodiment, a method of treating bruising or treating or preventing a disease associated with bruising can include administering to a patient in need thereof at least one topical formulation disclosed herein.

In an embodiment, diseases associated with bruising can include at least one of Senile Purpura and hemosiderin staining.

An embodiment of the present subject matter is directed to a method of treating a bruise, comprising administering to a patient in need thereof a therapeutically effective amount of topical formulation.

The topical formulation may be used with any known delivery system, such as an ointment, cream, lotion, liquid, roll-on, patch, gel, paste, micelle, dermal patch, or an occlusion. Occlusion can be achieved by applying a liquid form of the topical formulation to a dressing, such as an adhesive gauze pad or strip, to provide a medicated dressing and then applying the medicated dressing to the bruise. In an embodiment, the composition may be an ointment and the occlusion may be a cotton free adhesive layer. The topical formulation may further include one or more penetration enhancers, denaturants, pH regulators, exfoliates, stratum corneum penetrators, and/or keratolytic agents.

In an embodiment, the topical formulation, e.g., preservative-free formulations, may be applied to a dressing, securely wrapped, and gamma irradiated prior to use.

In an embodiment, a topical formulation for use in treating very sensitive skin may instead be applied continuously to a patient having sensitive or non-sensitive skin. The treatment time may be extended to achieve bruise reduction.

In an embodiment, the topical formulation for treating bruising may be prepared by mixing the preservative with water to form a first solution, adding citric acid to the first solution to form a second solution, adding butanol to the second solution to form a third solution, adding DMI to the third solution to form a fourth solution, and adding IPA to the fourth solution to form a fifth solution. An appropriate amount of TEA can be added to the fifth solution to provide a titrated solution having a pH ranging from about 3.8 to about 4.2. Water may then be added to the titrated solution to provide the final formulation.

In an alternative embodiment, the appropriate amount of TEA can be added to the fifth solution to provide a titrated solution having a pH of about 4.0.

In an alternative embodiment, the topical formulation for treating bruising may be prepared by mixing citric acid with water to create a first solution. IPA and DMI may be mixed to create a second solution. Optionally, 2-butanol and/or SLES can be mixed into the second solution. The first solution and the second solution may then be mixed thoroughly, creating a third solution. If desired, BKC may then be mixed into the third solution. The pH of the third solution may then be adjusted to a pH ranging from about pH 3.8 to about 4.2, e.g., 4.0, by dropwise addition of TEA. The weight of the solution may then be raised to about 94.0% by addition of an appropriate base. For a liquid formulation, the appropriate base may be water. For an ointment, the appropriate base may be a suitable ointment. As a final step, the pH may once again be checked, and if necessary, TEA may be added to arrive at a final pH of about 3.8 to about 4.2. Optionally, the final pH may be about 4.0.

### Example 1

### Testing of Sterile Topical Formulations for Sensitive and Normal Skin

Patients suffering from purpura were selected and their skin sensitivity was assessed. Each patient's purpura, or bruises, were categorized based on size. A small bruise was between about 1 and 2 cm in diameter. A medium bruise was between about 2 and 4 cm in diameter. A large bruise was between about 4 cm and about 8 cm in in diameter. The sterile topical formulations for patients with sensitive and normal skin were used as appropriate for each patient. An amount ranging from about 6 to about 8 drops of the topical formulation was applied to a small bruise. An amount ranging from about 12 to about 16 drops of the topical formulation was applied to a medium bruise, and an amount ranging from about 20 to about 24 drops of the topical formulation was applied to a large bruise. The topical formulation was first applied to a dressing, which was then placed over the bruised area of the skin. The occlusion provided by the dressing may be improved by treating (wrapping) the dressing with Coban. The dressing was left occluding the skin for about 8 to 12 hours. The dressing was re-applied daily until the purpura was at least 90% resolved. This test demonstrated that the topical formulation was effective at resolving purpura within two to five days of commencing treatment (Table 1).

TABLE 1
In Vivo Testing of Topical Formulation

| Age | Sex | Days to 90% Resolution | History | Medications |
|---|---|---|---|---|
| 84 | F | 2 | N/A | ASA |
| 78 | F | 5 | N/A | N/A |
| 72 | M | 4 | Atrial Fibrillation | Xeralto & |
| | | | | ASA |
| 84 | F | 5 | N/A | N/A |
| 71 | M | 4 | Cardiac Stent | Plavix |

### Example 2

### Testing of Sterile Topical Formulations for Sensitive, Non-Sensitive, and Very Sensitive Skin

The general procedure of Example 1 was repeated, but modified to address patients' skin sensitivity. When treating patients with very sensitive skin, the ointment including the topical formulation was applied directly to the skin, gently rubbed for 10 to 15 seconds, and covered by an adhesive dressing with the cotton pad removed, allowing for direct contact between the ointment and the plastic dressing. The dressing was left in place for 12 hours, then removed. The ointment was reapplied with gentle rubbing and without any occlusion. The ointment was applied for four hours up to three times daily, and treatment was continued until either bruise resolution (defined as at least 90% reduction in bruise size) or the appearance of substantial side effects that interfered with continuation of treatment. This treatment generally lasted less than six days. Exemplary data from this test are provided in Table 2.

TABLE 2
Selective Test Results

| Age | Sex | Skin Type | Contact Time to 90% resolution |
|---|---|---|---|
| 77 | F | Non-Sensitive | 32.5 Hours |
| 71 | M | Sensitive | 32.75 Hours |
| 84 | F | Non-Sensitive | 28.33 Hours |
| 72 | M | Sensitive | 42.75 Hours |
| 70 | M | Very Sensitive | 48.5 Hours |
| 66 | M | Very Sensitive | 52.25 Hours |

### Example 3

### Test Results

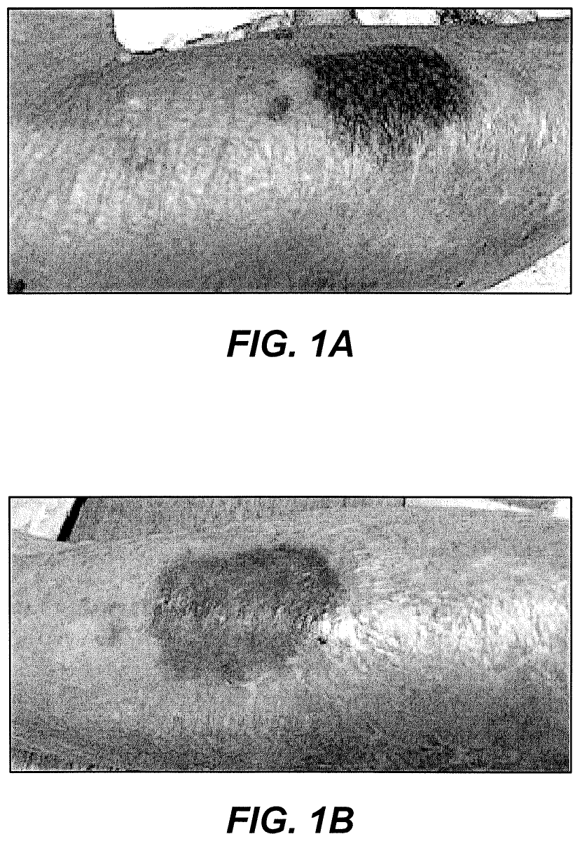

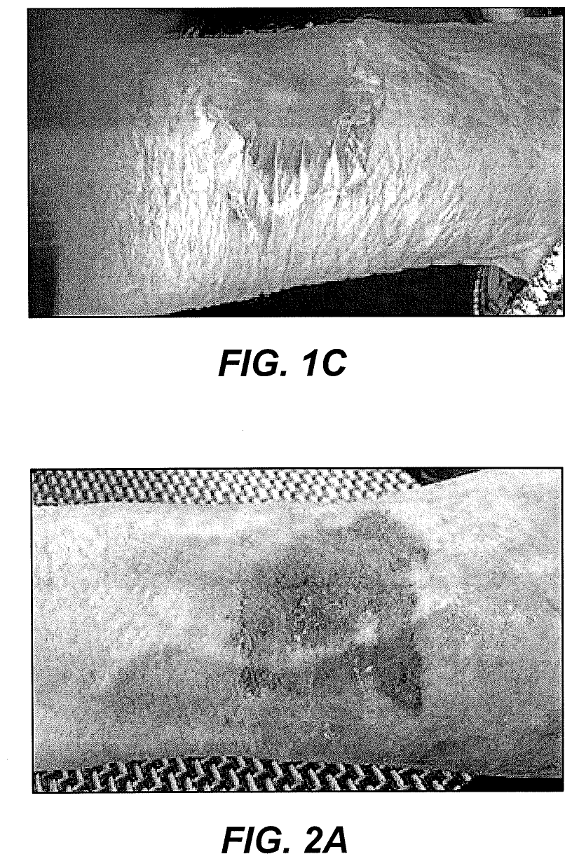

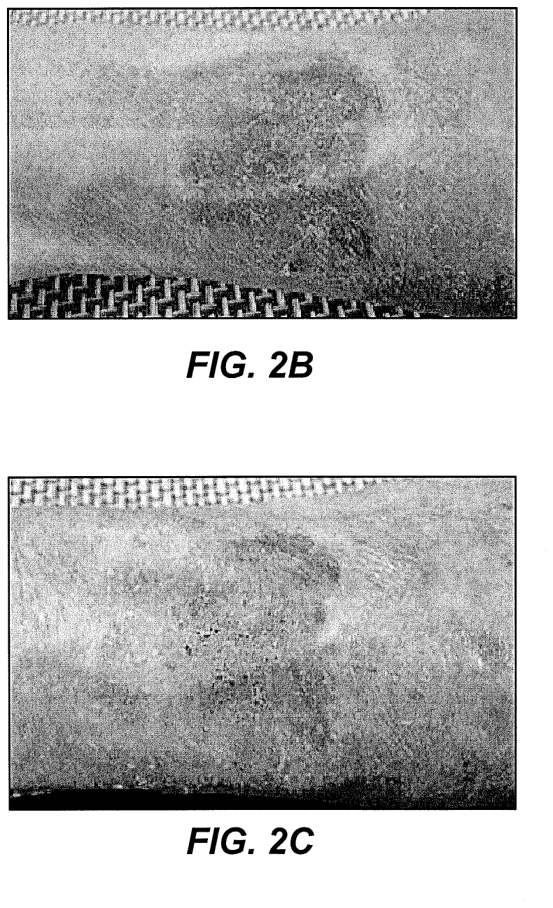

The procedure described in Example 1 was performed on two test subjects, each with a bruise on their left forearm. The bruising present pre-treatment is shown in FIGS. 1A and 2A. The reduction of bruising after three days of treatment with a topical formulation for treating bruising may be seen in FIGS. 1B and 2B. Significant resolution of bruising after five days and six days, respectively, is shown in FIGS. 1C and 2C.

### Example 4

### Testing of General Formulations

Three patients suffering from purpura skin were treated with the first embodiment of the general formulation. For this test, patient skin sensitivity prior to treatment was not considered. Patients with bruising on the arm received 1 cc of the first embodiment of the general formulation. A patient with a small bruise on the hand received 0.5 cc of the first embodiment of the general formulation. The general formulation was first applied to a dressing, which was then placed over the bruised area of the skin. The bruising was about 90% resolved or healed within a time period ranging from about 35 hours to about 52 hours.

A patient suffering from purpura skin was treated with the second embodiment of the general formulation. For this test, patient skin sensitivity prior to treatment was not considered. The patient received 0.5 cc of the second embodiment of the general formulation. The general formulation was first applied to a dressing, which was then placed over the bruised area of the skin. The bruising was about 90% resolved or healed within about 112 hours. The results are summarized in Table 3 below.

TABLE 3

| | | Contact | | |
|---|---|---|---|---|
| | | Time | | |
| | | to 90% | | |
| Age | Sex | resolution | Formula | Profile |
| 71 | M | 45 Hours | General | taking Xeralto, blood pressure |
| | | | | medication and |
| | | | | cardiac medication |
| 67 | M | 35 Hours | General | taking blood thinners and |
| | | | | blood pressure |
| | | | | medication |
| 52 | M | 52 Hours | General | taking diabetes medication |
| | | | | and ASA for cardiac |
| | | | | stent |
| 60 | F | 112 Hours | Alternative | taking Plavix and |
| | | | | blood pressure medication |

It is to be understood that the topical formulation for treating bruising is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. A topical formulation for treating bruising, comprising citric acid, 2-butanol, dimethyl isosorbide, triethanolamine, and water.

2. The topical formulation for treating bruising according to claim 1, wherein the formulation comprises from about 0.1% to about 10.0% by weight citric acid, from about 0.1% to about 10.0% by weight 2-butanol, and from about 0.2% to about 15.0% by weight dimethyl isosorbide.

3. The topical formulation for treating bruising according to claim 1, wherein the formulation is in a topical administration form selected from the group consisting of an ointment, a cream, a lotion, a liquid, a roll-on, a patch, a gel, a paste, a micelle, a dermal patch, or an occlusion.

4. The topical formulation for treating bruising according to claim 1, further comprising at least one of a preservative, sodium laureth sulfate, isopropyl alcohol, a humectant, a scented oil, and a base.

5. The topical formulation for treating bruising according to claim 4, wherein the formulation comprises about 1.0% by weight citric acid, about 0.3% by weight 2-butanol, about 30.0% by weight isopropyl alcohol, about 10.0% by weight dimethyl isosorbide, and about 0.5% by weight of the preservative.

6. The topical formulation for treating bruising according to claim 4, wherein the formulation comprises about 1.0% by weight citric acid, about 0.2% by weight 2-butanol, about 5.0% by weight isopropyl alcohol, about 10.0% by weight dimethyl isosorbide, and about 0.5% by weight of the preservative.

7. The topical formulation for treating bruising according to claim 4, wherein the formulation comprises about 10.0% by weight citric acid, about 10.0% by weight 2-butanol, about 1.0% by weight sodium laureth sulfate, and about 15.0% by weight dimethyl isosorbide.

8. The topical formulation for treating bruising according to claim 7, wherein the formulation comprises about 0.5% by weight of the preservative.

9. The topical formulation for treating bruising according to claim 4, wherein the composition comprises isopropyl alcohol, and sodium laureth sulfate.

10. The topical formulation for treating bruising according to claim 9, wherein the formulation comprises about 1.0% by weight citric acid, about 0.5% by weight 2-butanol, about 0.5% by weight sodium laureth sulfate, about 20.0% by weight isopropyl alcohol, and about 10.0% by weight dimethyl isosorbide.

11. The topical formulation for treating bruising according to claim 10, wherein the formulation comprises about 0.5% by weight of the preservative.

12. The topical formulation for treating bruising according to claim 4, wherein the formulation comprises isopropyl alcohol.

13. The topical formulation for treating bruising according to claim 12, wherein the formulation comprises about 1.0% by weight citric acid, about 0.2% by weight 2-butanol, about 20.0% by weight isopropyl alcohol, and about 10.0% by weight dimethyl isosorbide.

14. The topical formulation for treating bruising according to claim 13, further comprising about 0.05% by weight of the preservative.

15. A topical formulation for treating bruising, comprising an exfoliant, a protein denaturant, a penetration enhancer, triethanolamine, water, and at least one of a preservative, a detergent, a skin denaturant, and a base.

16. A method of treating bruising, comprising administering the topical formulation of claim 15 to a patient in need thereof.

17. The method of treating bruising of claim 16, wherein the formulation comprises about 1.0% by weight citric acid, about 0.3% by weight 2-butanol, about 30.0% by weight isopropyl alcohol, about 10.0% by weight dimethyl isosorbide, and about 0.5% by weight of a preservative.

18. The method of treating bruising of claim 16, wherein the formulation comprises about 10.0% by weight citric acid, about 10.0% by weight 2-butanol, about 1.0% by weight sodium laureth sulfate, and about 15.0% by weight dimethyl isosorbide.

19. The method of treating bruising of claim 16, wherein the formulation comprises about 1.0% by weight citric acid, about 0.5% by weight 2-butanol, about 0.5% by weight sodium laureth sulfate, about 20.0% by weight isopropyl alcohol, and about 10.0% by weight dimethyl isosorbide.

20. The method of treating bruising of claim 16, wherein the formulation comprises about 1.0% by weight citric acid, about 0.2% by weight 2-butanol, about 20.0% by weight isopropyl alcohol, and about 10.0% by weight dimethyl isosorbide.
